(19) United States
(12) Patent Application Publication
(10) Pub. No.: US 2006/0127500 A1
(43) Pub. Date: Jun. 15, 2006

(54) Zinc oxide in the form of an improved flow powder, method for the production and use thereof in polymers

(76) Inventors: Tomizio Guidi, Seraing (BE); Jules Polart, Morlanwelz (BE)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 10/529,301

(22) PCT Filed: Sep. 24, 2003
(86) PCT No.: PCT/IB03/05612

§ 371 Date: Mar. 25, 2005

(30) Foreign Application Priority Data
Sep. 25, 2002 (BE) 2002/0557

(51) Int. Cl. A61K 33/32; C03C 8/04; C08K 5/09

(52) U.S. Cl. 424/641; 524/399; 501/26

(57) ABSTRACT

A method is disclosed for atomizing an aqueous suspension of zinc oxygen compounds using a nozzle in a flow of hot air enabling a powder to be obtained, the Flodex index thereof being less than 15 and the compressibility index thereof being less than 20%. The powder is formed from micro-spherical particles whose diameter is approximately 50-200 μm, preferably 70-90 μm, made of smaller unit particles which are perfectly dispersable in elastomers or paints, for example. The products can be used advantageously in the vulcanization of rubber.

The present invention concerns oxidised zinc compounds in the form of powder with improved pourability, the method of manufacturing these and their use in the field of polymers, in particular elastomers.

Throughout the whole of the following text, oxidised compounds means here zinc oxides, hydroxides, carbonates or hydroxycarbonates and derivatives thereof.

Zinc oxide is used in very varied fields, such as the fields of enamels, varistors, oils, paints, various reagents for polymers, both plastics and elastomers, and even in animal feeds. For these many applications, it is in various forms, generally in the form of a powder. In the specific field of elastomers, zinc oxide is employed in particular as a sequestering catalyst for the vulcanisation reaction, during the cross-linking of the elastomers, or as a semi-reinforcing filler:

as a catalyst for the cross-linking reaction (that is to say the creation of a three-dimensional bridge lattice by virtue of a vulcanisation agent, such as sulphur), the zinc oxide is combined with stearic acid in order to form an activating complex soluble in gum, which combines with the vulcanisation agent to allow the fixing of the macro-molecular chains of the elastomer; the mechanisms of actions are transient (not very stable complexes) and/or ill defined in the case of variations in certain parameters (type of zinc oxide, specific surface, etc);

as fillers, in the same way as other powdery products (carbon black, chalk, kaolins, talc, etc) it confers specific properties on the product (conductivity, improvement in resilience, etc).

The physical and chemical parameters of the zinc oxide have an influence on the behaviour of the product in the great majority of applications. In order to quantify the differences between the various zinc oxides the producers normally use physical and chemical parameters which allow their classification, in particular:

the BET expressed in m²/g of product (indicative of the specific surface)

the residual heavy metal contents (indicative of the purity).

The BET specific surface value measured corresponds to the accessible surface of the product for a given chemical compound. This surface varies according to the nature (gas or liquid) of the solvent used for the measurement and therefore remains an indicative value. It is generally accepted that products with a high BET have an improved surface chemical activity (reactivity) but difficult dispersion. There are currently commercially available zinc oxides whose BET specific surface varies over a wide range of values, between 0.5 m²/g and 100 m²/g approximately.

The specific surface being considered to be a particularly influential variable in the reactivity of the ZnO, manufacturers use various synthesis methods which make it possible to obtain oxides with different physical natures, according to their subsequent application.

One of the manufacturing methods is the so-called “wet” method (wet process). It comprises two main phases:

Phase 1: the reaction of SO₂ on a metallic zinc, in the presence of water, produces ZnS₂O₄, and is followed by a precipitation by an alkaline agent (NaOH) in order to give a precipitated zinc oxide or zinc hydroxide, which is next filtered and then washed.

It is during the precipitation reaction that it is possible firstly to control the BET of the product (in particular according to the precipitation rate, the nature of the alkaline agent, the concentration of the products, etc). This is because the conditions for obtaining a zinc oxide or zinc hydroxide cake govern the final nature of the product (the form of the particles, specific surface, etc). These parameters can also be modified during subsequent processing operations, but the majority of the conventional specific characteristics stem from this first complex reactive phase.

Phase 2: in this phase the calcination of the zinc hydroxide or zinc oxide is carried out, or the drying of the oxide issuing from the filtration cake, an oxide which is then packed in bags or hoppers.

The drying can be carried out:

either in rotary ovens where the product is in direct contact with the flame or with the hot walls of the oven, giving rise to large very hard granules, which then need to be ground before use. The zinc oxide thus obtained often has a low specific surface;

or by sucking the dense compact filtration cake in a flow of hot air, according to a so-called “flash” process. The product obtained is a very fine powder, sometimes having agglomerates which require grinding before dispersion.

The choice of the drying methods, as of its parameters (temperatures, speed of fluids, type of insulation, etc) makes it possible to obtain specific products.

However, these drying techniques result in zinc oxides which have mediocre pourability, frequently having agglomerates and setting during storage. These agglomerates are also detrimental to good incorporation of the said oxides in the elastomers, but also during conveying thereof, where they frequently block the pipes.

The inventors surprisingly discovered that, by using for the drying of the oxidised compounds of zinc, in particular zinc oxide, an atomisation process, and by precisely adjusting certain operating parameters, it was possible to obtain an oxidised zinc compound with improved pourability and dispersibility, and with good reactivity, without altering its specific surface.

The present invention therefore concerns an oxidised zinc compound in the form of micro-spherules, characterised in that it has a Flodex index of less than 15, and preferably less than 10.

The Flodex index is a test for measuring the ease of flow of the powders. The apparatus used is a receptacle in which the powder to be tested is placed and whose base consists of a diaphragm with a calibrated opening through which it is observed whether there is any flow of the said powder. Through successive tests, the minimum diameter through which this powder flows freely is determined. This diameter (in millimetres) corresponds to the Flodex index.

A very low index (less than 15, or even less than 10) therefore indicates a product with very high pourability, referred to here as “free flowing”. The many advantages which stem therefrom are in particular:

facilitated bagging;

improvement to the apportioning conditions in semi-automatic and automatic systems, mainly with regard to reproducibility, the apportioning rates and the elimination of blockages and clumping effects;

the elimination of stagnation of powder in pipework elbows during pneumatic conveying for example;

limitation to the emission of fines during various handling operations (for example during apportioning, filling of tanks, etc), which appreciably reduces problems related to safety, the environment and losses of product.

Preferably, the apparent density of the oxidised zinc compounds in powder form accorded to the invention is greater than approximately 0.8. In addition, they advantageously have a compressibility index [(packed density−apparent density)×100/packed density] of less than approximately 20%, preferably less than approximately 15% and advantageously between approximately 10% and 12%.

The oxidising compound in powder form can thus be stored over a long period (at least a year), without exhibiting agglomerates or any phenomenon of “clumping”.

The oxidised zinc compounds are preferably chosen from amongst a zinc oxide, a zinc hydroxide, a zinc carbonate, a zinc hydroxycarbonate or a mixture of these.

The present invention also concerns a method of preparing the aforementioned oxidised zinc compound, characterised in that it consists of injecting, by means of a nozzle, an aqueous suspension of the said oxidising compound, having a solid matter content of 25% to 70% by weight, and a pressure of 10 to 100 bar approximately, within an atomisation chamber, in a gas flow entering at a temperature of 250° to 800° C. approximately and leaving at a temperature of 50° to 300° approximately.

The aqueous suspension of oxygenated compound of zinc at 25-70% by weight solid matter, preferably 35% to 60% by weight, is sufficiently fluid to be sprayed (atomised) by means of a nozzle.

The solid matter content is preferably between 40% and 45% by weight in the aqueous suspension.

The drying technique according to the invention allows the addition of certain additives in the suspension and makes it possible to obtain perfect homogenisation within the product. In particular, if it is wished to increase the solid matter content up to 45-70% by weight, it may be advantageous to add a dispersing agent stabilising the said suspension, such as a polyacrylate.

The gas flow may be air, nitrogen or any suitable gas (issuing from recycling, for example from a combustion product). The gas stream is preferably a stream of air.

The residence time in the atomisation chamber does not exceed 10 seconds, and is preferably around 4 to 5 seconds.

The BET specific surface of the product obtained may range up to 150 m²g, but is advantageously less than 100 m²g and preferably less than 50 m²g.

The method according to the invention, referred to as atomisation, is a drying process which preserves all the physical and chemical properties (in particular the specific surface) of the particle as from the precipitation reaction, whilst modifying the macroscopic structure of the product by assisting the formation of micro-spherules with a close granulometric distribution. This is because it is clear that the micro-spherules have a D₅₀ (size corresponding to 50% of the total granulometric distribution curve) of between approximately 50 and 200 μm, advantageously between approximately 50 and 100 μm, and preferably between approximately 70 and 90 μm.

In order to obtain a narrow distribution of the granularity of the particles, for introducing the suspension of oxidised zinc compound into the atomisation chamber, an injection nozzle is preferable to a rotating disc.

This is because, in order to obtain coarser particles, with a rotating disc, it is necessary to reduce its rotation speed. The droplets projected are then too coarse to be dried before reaching the walls of the chamber and cause depositions on them.

In the present invention, in combination with the adjustment of the pressure, the choice of the diameter of the discharge from the spray nozzle (for example between 0.8 and 6 mm) makes it possible to vary the size of the required micro-spherules, between approximately 50 and 100 μm, always producing them in a narrow granulometric distribution.

Advantageously, the injection pressure is between 15 and 80 bar approximately. The nozzle used is preferably a spray nozzle with a vortex chamber.

The oxidising compound obtained by the method according to the invention no longer has any “clumping” and makes it possible to obtain a flow of the “micro-ball” type, which makes the product very easy to convey through pneumatic channels without clogging the pipes.

This is an important advantage in the use of the oxidised zinc compounds according to the invention in industrial mixers with pneumatic migration of the powder ingredients, and for their use in polymer matrices, preventing the formation of invisible agglomerates during the mixing phase.

It was possible to reveal these advantages particularly during the vulcanisation of elastomers. Thus better dispersion in particular of zinc hydroxycarbonate and zinc oxide was found in rubber matrices with reduced mixing times, and better reproducibility (which has an effect on parameters such as the degree of cross-linking, rupture strength and yield strength), as well as an appreciably reduced degree of fouling of the moulds, making it possible to use these products over a longer period. In addition, after storage, the absence of whitish bloom, frequently present on the surface of rubber with the zinc oxides of the prior art, was noted.

This better dispersion is due to the fact that the micro-spherules consist in reality of smaller unitary particles having a D₅₀ of between 1 and 20 μm approximately, advantageously between 1 and 15 μm, without binder.

When the zinc compound is incorporated in the elastomer matrix, or put in suspension in a liquid (water for example), the micro-spherules disintegrate, giving rise to these unitary particles which are smaller but have an unchanged BET specific surface.

With zinc oxide, these unitary particles have a D₅₀ which is rather between 1 and 10 μm approximately, preferably between 1 and 5 μm, or even between 1 and 3 μm approximately. With zinc hydroxycarbonate, the dimensions (D₅₀) of these unitary particles are between approximately 1 and 20 μm, and preferably between 1 and 15 μm.

A binder, such as polyol, could nevertheless be added to the oxidised zinc compound in order better to bind the unitary particles to each other, forming the micro-spherule. However, such a binder is not necessary in the invention and makes it possible not to “contaminate” the material in which the micro-spherules will be incorporated (elastomers, etc.).

The oxidised zinc compounds according to the present invention can be produced by the method described above, finding an advantageous use in the cross-linking of polymers, in particular in the vulcanisation of elastomer.

These compounds can also be used as pigments or fillers, for example in paints, glass or ceramics.

The present invention will be illustrated by non-limiting examples, with reference to the accompanying figures, in which:

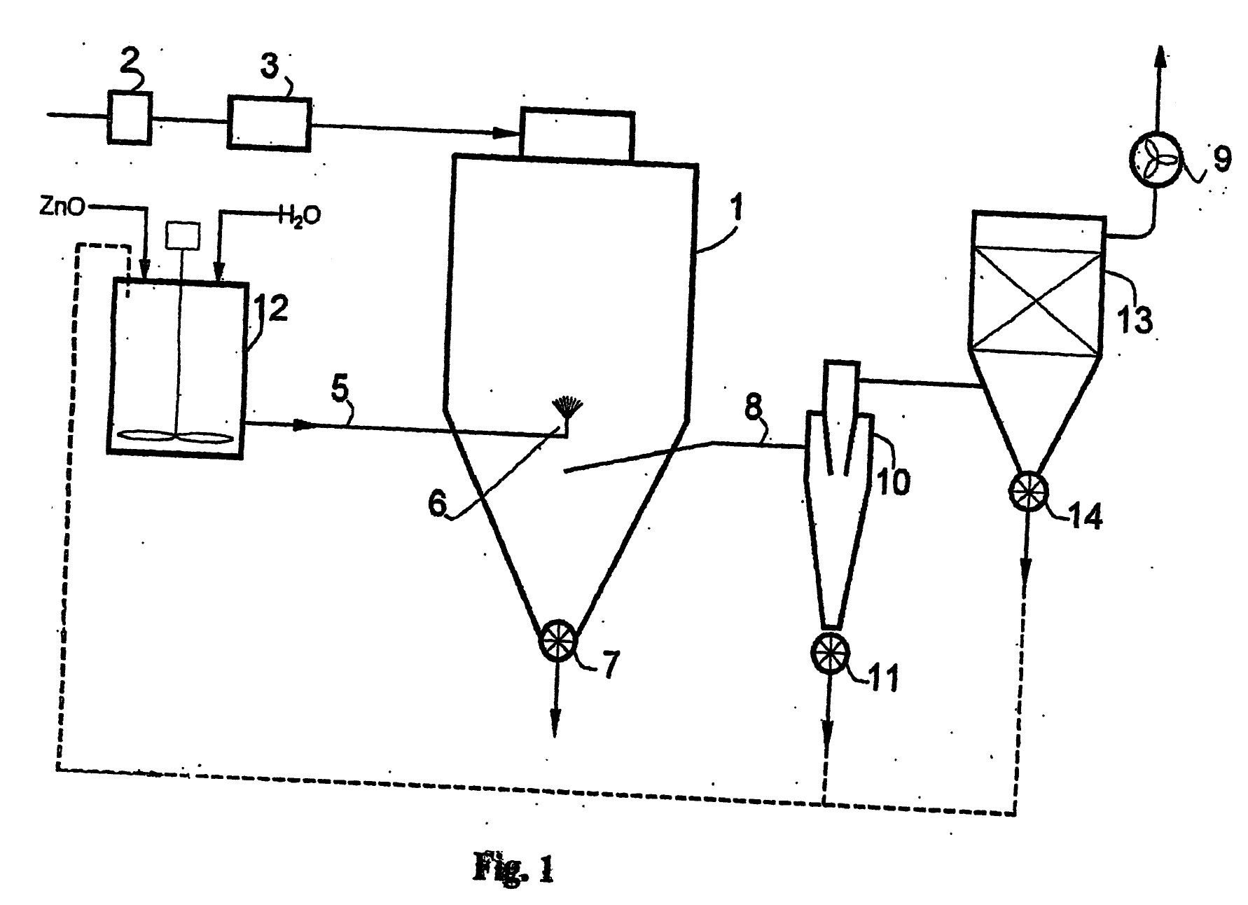

FIG. 1 is a diagram presenting the device for implementing the method according to the invention,

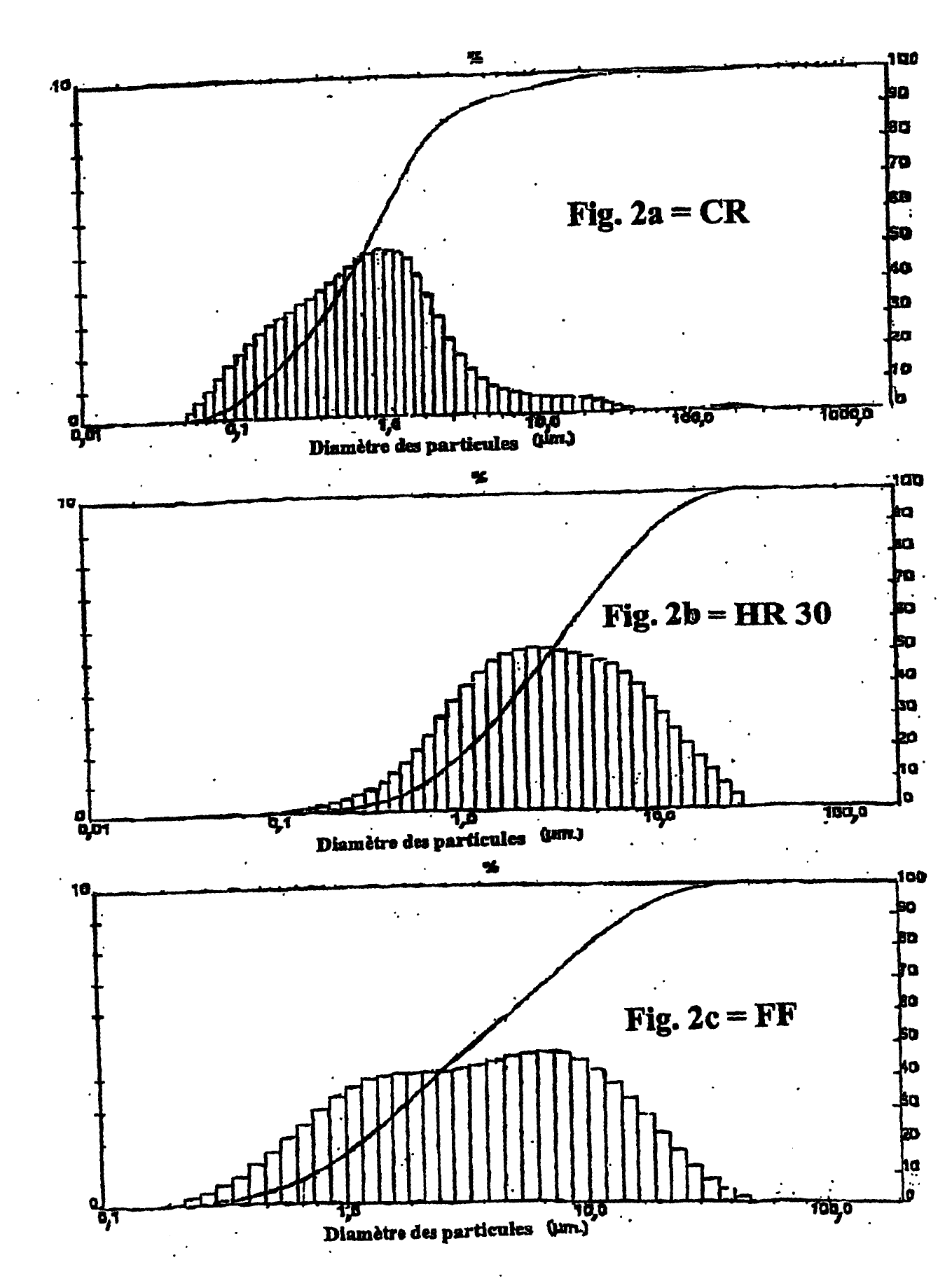

FIGS. 2a, 2b and 2c show the granulometric distribution of several types of zinc oxide,

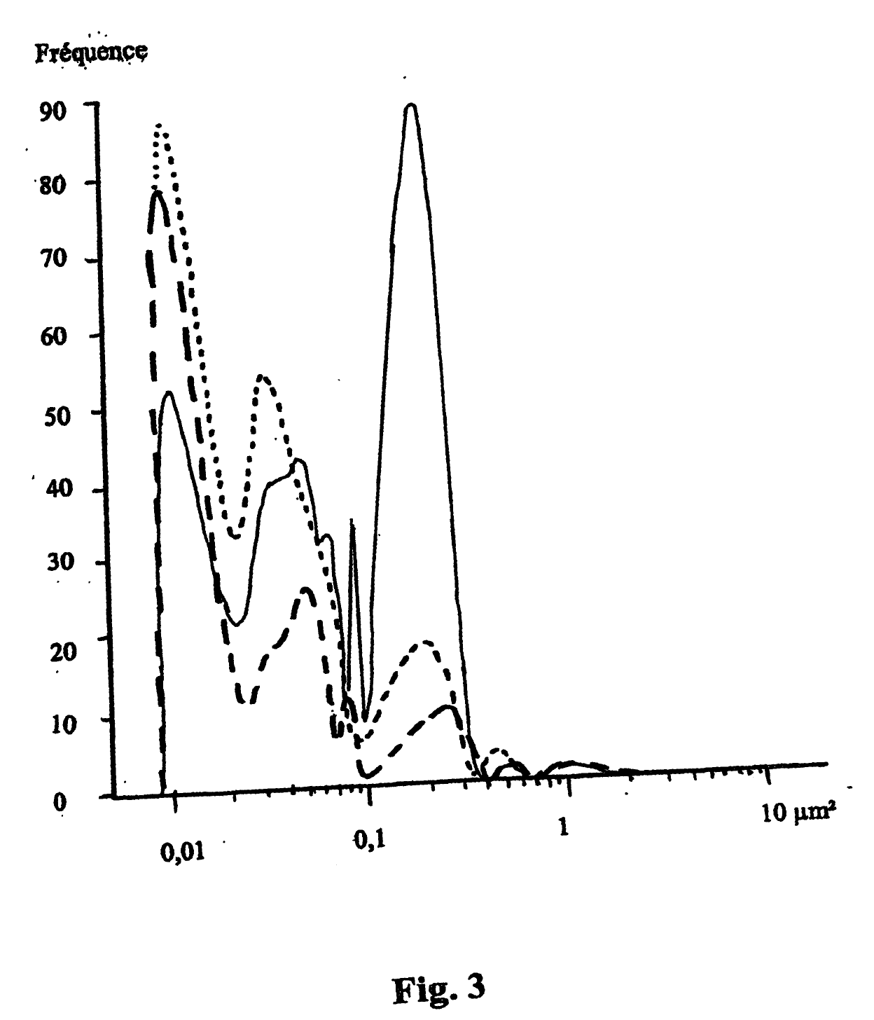

FIG. 3 presents curves showing the frequency of appearance of the particles according to their cross section on sections through rubber matrices incorporating various zinc oxides,

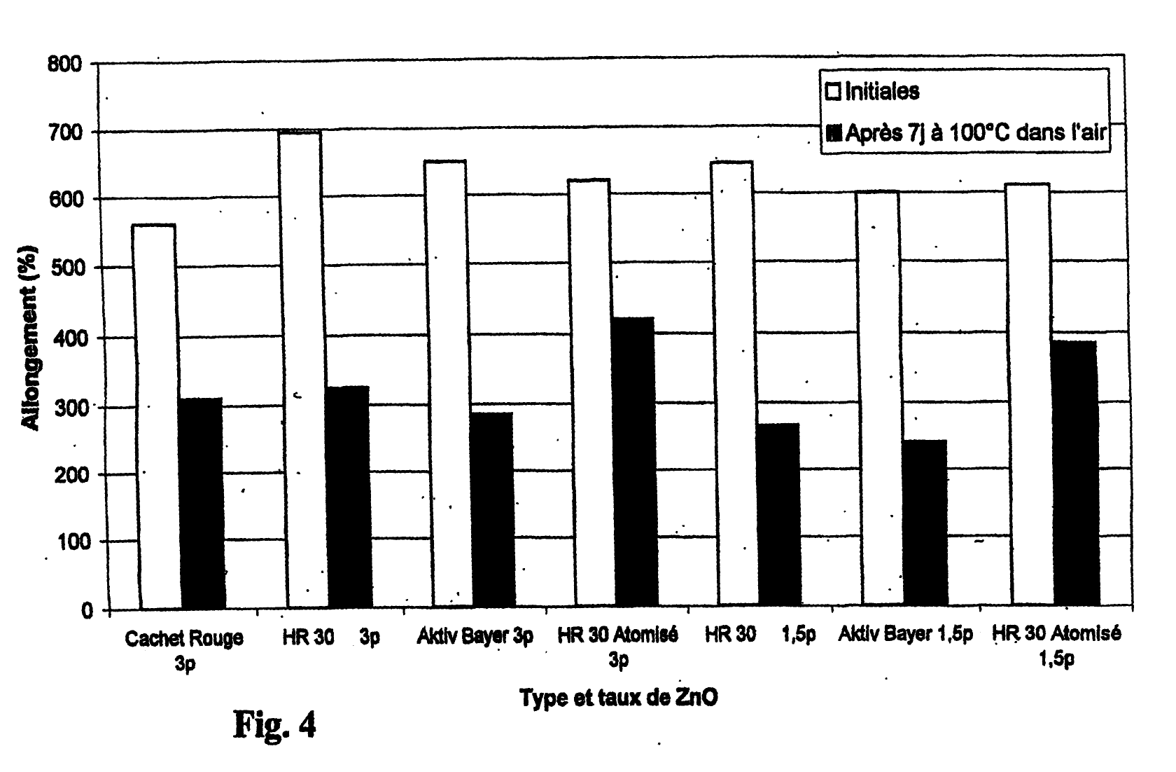

FIG. 4 is a graph showing the yield strength of rubber pieces in which various zinc oxides have been incorporated,

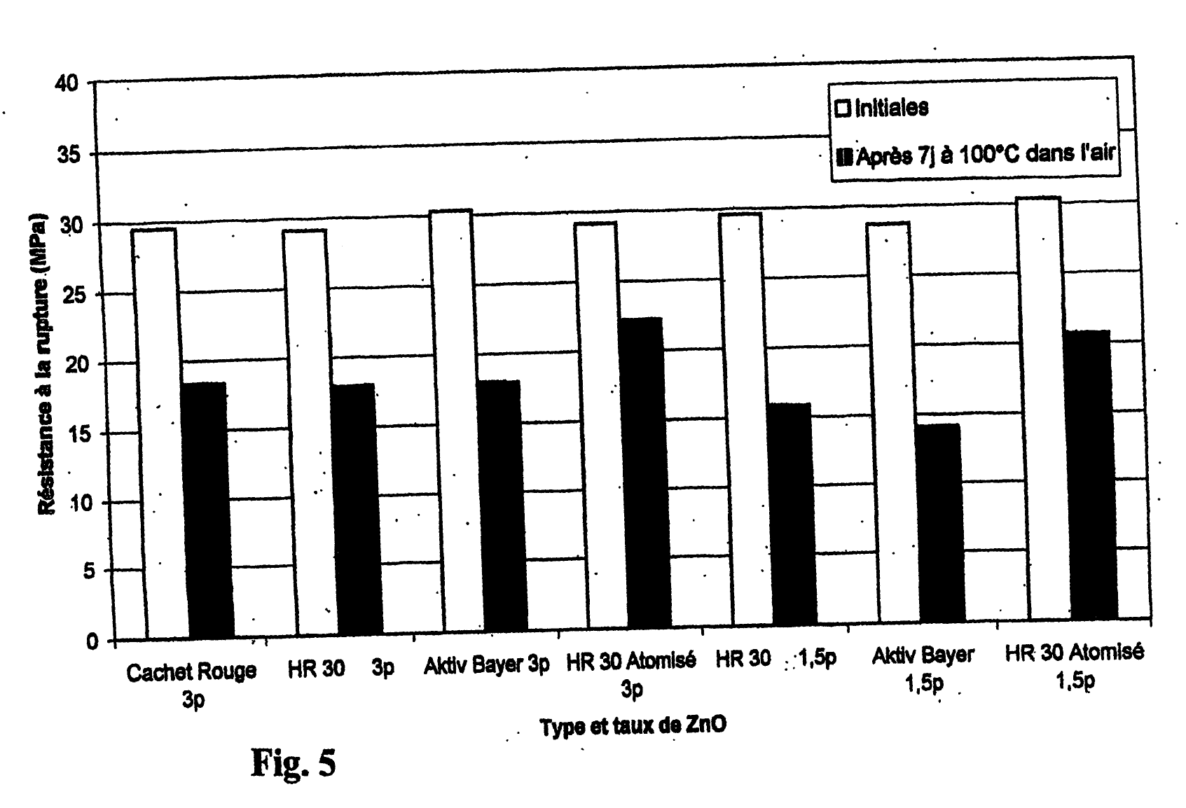

FIG. 5 is a graph showing the yield strength of rubber pieces in which various zinc oxides have been incorporated,

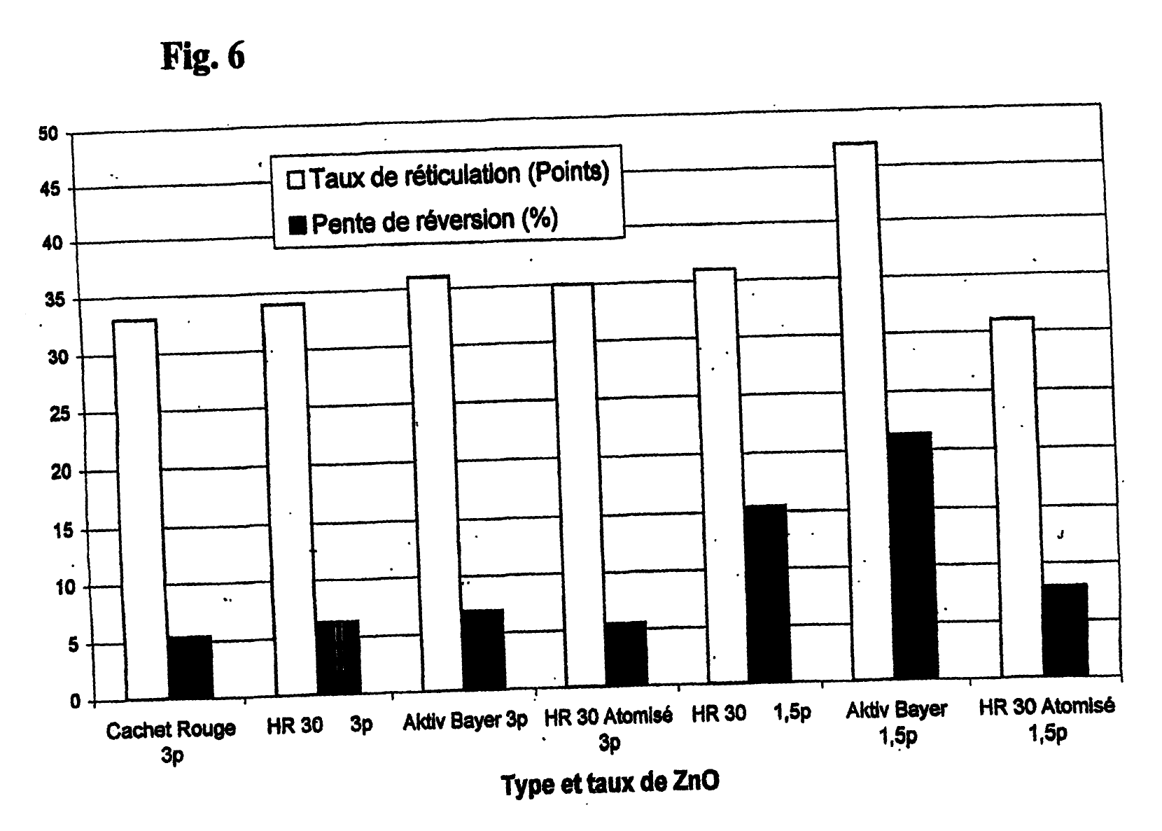

FIG. 6 is a graph showing the degree of cross-linking and the reconstitution slopes of rubber matrices in which various zinc oxides have been incorporated,

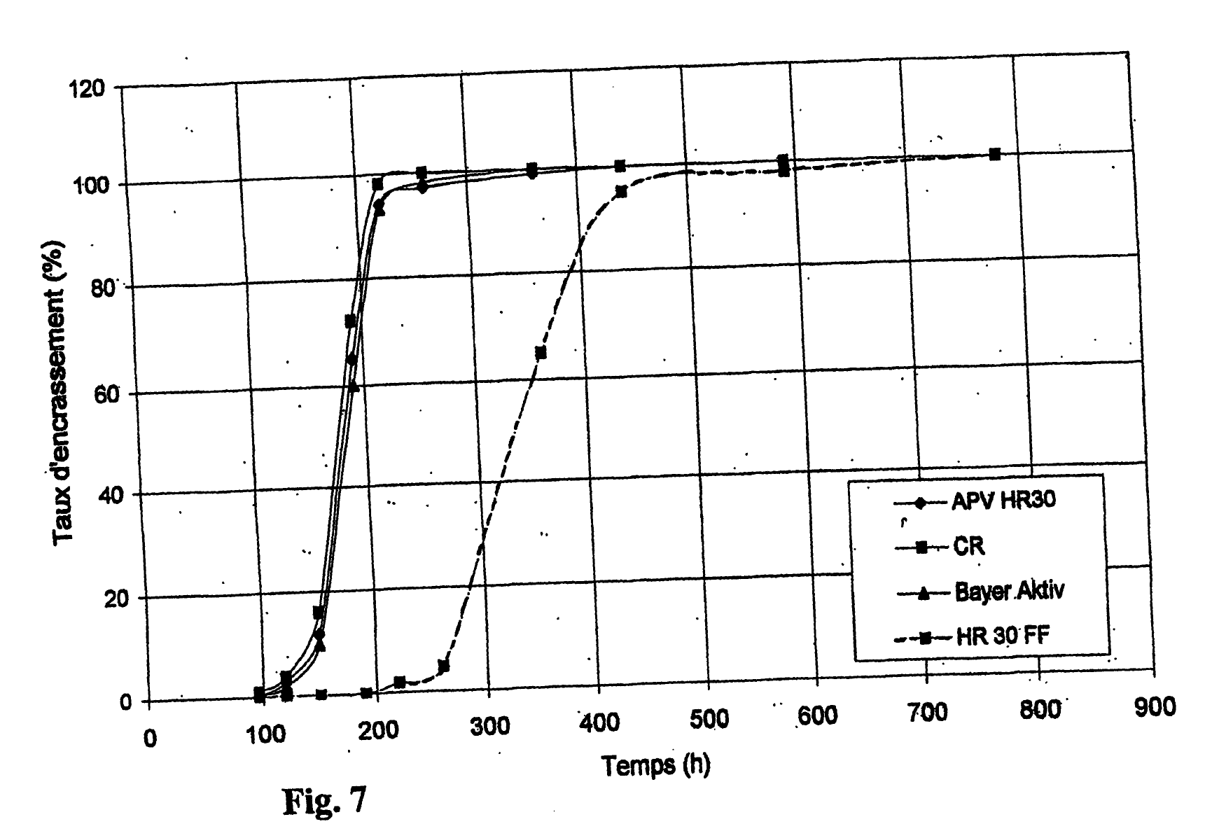

FIG. 7 is a graph showing the rate of fouling of moulds used for the manufacture of rubber pieces incorporating various zinc oxides.

### EXAMPLE 1

### Device

A device for implementing the method according to the invention presented in FIG. 1 comprises a cylindrical atomisation chamber 1 with a conical bottom in which a stream of hot air circulates. The incoming air first passes through a filter 2 and a burner 3 for preheating it and, at a temperature of 550° C., enters the top part of the atomisation chamber 1 through a distributor with fins 4. The air flow during the test was around 700 N m³/h.

The zinc oxide is applied through the pipe 5 by means of a pump, in the form of an aqueous suspension containing 40% to 45% by weight ZnO prepared in a mixer-disperser 12. The said suspension is introduced, at a rate of approximately 80-100 litres per hour at a pressure of 20 to 30 bar, by means of a nozzle 6 disposed at the centre of the chamber 1. The said suspension is sprayed (atomised) and dried in a few seconds in the stream of hot air.

The particles of ZnO obtained fall into the bottom part of the chamber where they are collected and discharged through the valve 7 in order for example to be bagged immediately. At this level between 85% and 90% of the FF (free flowing) zinc oxide is collected in the form of spherules with a mean diameter between 70 and 100 μm containing less than 0.5% residual water. The finest particles are discharged in the air leaving the chamber through the pipe 8 under the suction action of a fan 9. They are then separated from the emerging air for example in a cyclone 10 and recovered by the valve 11 in order possibly to be recycled in the preparation of the suspension in the disperser 12. Approximately 10% to 15% by weight fines (non agglomerated power in the form of spherules) are thus immediately separated from the zinc oxide spherules constituting the FF ZnO of the invention, and recycled.

The air is filtered by means of a bag filter 13 before leaving the circuit. Any particles recovered at this stage are collected at the valve 14 and can also be recycled to the disperser 12 (along the broken line).

The nozzle 6 used can be a spray nozzle of the type having a vortex chamber (of the Delavan SDX type) with an exit diameter of 1.62 mm in the example presented, or a dual-fluid nozzle in which the energy is afforded by the compressed air, which makes it possible to obtain very high degrees of atomisation and therefore finer powders.

The following Table 1 presents the results of analysis of two series of zinc oxide dried by the atomisation method according to the invention by means of the device described above.

ZnO no 1 is a product issuing directly from the filtration cake of the so-called “wet” preparation method.

ZnO no 2 is a zinc oxide obtained by calcination.

These two oxides were put in suspension in water (40%-45%) and were subjected to the method according to the invention in a device like the one presented in Example 1.

TABLE 1

| | ZnO n° 1 | ZnO n° 2 |
|---|---|---|
| BET surface (m²/g) | 38 | 7.2 |
| Oil absorption | | |
| Ml/100 g | 51.5 | 40.8 |
| Max. couple (mNm) | 360 | 180 |
| Pourability | | |
| Flodex index (mm) | 5 | 5 |
| Density | | |
| apparent | 0.893 | 1.128 |
| packed | 1.010 | 1.263 |
| Compressibility Index | 11.6 | 10.7 |
| Granulometry | | |
| Total not passed (%) 212 μm | 0.4 | 0.2 |
| 106 μm | 66.4 | 56.3 |
| 75 μm | 87.6 | 81.4 |
| 45 μm | 97.8 | 96.4 |
| 32 μm | 99.7 | 99.4 |
| Passed 32 μm | 0.3 | 0.6 |
| Not passed wet (%) 45 μm | <0.1 | <0.1 |
| Chemical composition | | |
| H₂O at 105° C. (%) | 0.24 | 1.08 |
| ZnO (%) | 95.6 | 98.2 |
| Sₜₒₜₐₗ (%) | 1.1 | 0.39 |
| S_{sulfur}(%) | 0.65 | 0.09 |
| S_{sulfate}(%) | 0.21 | 0.37 |
| Cu(ppm) | 3.3 | 3.0 |
| Mn(ppm) | 0.8 | 0.7 |
| Pb(ppm) | 15 | 16 |
| Cd(ppm) | <0.5 | <0.5 |

### EXAMPLE 2

### Comparison of Physical and Chemical Characteristics of Several Zinc Oxides

Table 2 below, associated with FIGS. 2a to 2c, makes it possible to compare the physical and chemical characteristics of zinc oxides dried by different methods

a. the conventional ZnO CR thermal method

b. the so-called “spin flash” Zno HR 30 method

c. the atomisation method according to the invention ZnO FF

(Free Flowing)

d. a zinc oxide dried by a “flash” method

TABLE 2

| | a | b | d | c | |
|---|---|---|---|---|---|
| ZnO examples | ZnO CR | ZnO HR30 | ZnO | ZnO FF | |
| Drying | thermal | Spin flash | flash | atomisation | |
| method | | | | | |
| BET (m²/g) | 5 | 46 (20-50) | 43 | 47 (40-45) | |
| Oil | 35 | 55 | 49 | 57 | |
| absorption | | | | | |
| (ml/100 g) | | | | | |
| Flodex index | 36 | 34 | 30 | 9 | |
| (mm) | | | | | |
| D10 | 0.15 | 0.73 | 0.73 | 0.79** | 60.1* |
| D50 | 0.78 | 2.95 | 3.18 | 3.85** | 79.1* |
| D99 | 28.36 | 23.45 | 33.2 | 32.75** | 95.1* |

*before dispersion
**after dispersion 15 minutes (here in water)
The values D represent the particle sizes (in μm) corresponding respectively to 10, 50 and 99% of the total distribution curve, by laser measurement.
There are found, after dispersion of ZnO FF (c) in water, particle sizes which are comparable to those of the other known products, without having to undergo a mechanical operation such as grinding (cf also the graph in FIG. 3).

### EXAMPLE 3

### Incorporation of Various Zinc Oxides in Solid Rubber Matrices

On a cylinder mixer heated to 150° C. (set temperature) the polymer is first of all heated for two minutes and then zinc oxide and stearic acid are introduced simultaneously. The mixing is continued for five minutes, before pressing the mixture in a plate press for two minutes at 150° C. Fine sections (50 nanometres) are prepared by ultramicrometry at −130° C. using a diamond blade, before being deposited on a copper grille with a view to observing them under a transmission electron microscope (TEM). The area of the section of particles observable on the section of the sample was measured.

The constituents and quantities used were as follows:

for 100 parts by weight of polymer (solid rubber):

15 parts by weight ZnO (CR, HR30 or FF having the properties presented in Table 2, Aktiv Bayer ZnO having a Flodex index >36 and a BET specific surface of around 45 m²/g)

3 parts by weight stearic acid.

For this test, the proportions of ZnO and stearic acid were increased compared to the conventional proportions (5 parts ZnO and 1.5 to 2 parts stearic acid) so as to be able to visually observe their behaviour in the rubber matrix.

FIG. 2 presents the comparisons of the results of analysis of the images for these three oxides: frequency according to the area in μm² of the section of the particles observable on the section. These curves show principally that the ZnO according to the invention (ZnO FF dried by atomisation) has, after incorporation in the rubber matrix, fewer large particles than the Zno CR (dried by a conventional thermal method): in fact a significant reduction in the peak around 0.02 μm² is noted. The profiles of the curves for ZnO FF and SnO HR30 are similar.

Nevertheless, in the elastomer mixture the number of small particles is higher for ZnO FF, which means that the reaction on page 1 line 14 of the products with each other is more complete and consequently the levels of ZnO normally used can be decreased very substantially (that is to say reduced from 5% to 2.5%, or even to 1.5% by weight).

### EXAMPLE 4

### Measurements of the Breaking Strength and Yield Strength

The tests on breaking and yield strengths were carried out in accordance with ISO 37, NF T46.002 and ASTM D 412 on the samples prepared in accordance with the protocol of Example 3 above, with NR rubber (Natural Rubber). The quantities of the various zinc oxides introduced into the matrices were 3 parts (3 p) by weight or 1.5 parts (1.5 p) by weight. The mechanical properties (breaking strength, yield, etc) were measured before and after exposure to air for seven days at 100° C. The results are presented in FIGS. 4 and 5.

These results show better resistance to aging of the rubbers containing 3 p and 1.5 p ZnO FF according to the invention.

Similar tests with EPDM (Ethylene Propylene Diene Monomer) matrices exhibited greater improvements.

### EXAMPLE 5

### Degree of Cross-Linking and Reconstitution Slope

On the samples of Example 4, the degrees of cross-linking (creation of “bridges” between the polymer chains) were measured graphically on the rheometric curves of the mixtures (cross-linking curves) at 170° C. on an oscillating-rotor rheometer (Type R 100). It is a question of the difference between the maximum couple (the high point of the curve) and the minimum couple (the low point of the curve). It is expressed in “points”.

With regard to the reconstitution slope, the decrease in the curve (in points) is measured graphically 6 minutes from the maximum couple. In this way the residual bridge density is tested after 6 minutes at the vulcanisation temperature.

It is clear that, with the ZnO FF according to the invention (cf. FIG. 6), the reconstitution is appreciably decreased, which avoids the use of additional products with anti-reconstitution action. The activation energy of the ridges created with ZnO FF is therefore greater than that of other types of zinc oxide.

### EXAMPLE 6

### Suitability for Moulding/Limitation of Fouling

The tests carried out on the industrial use of free flowing ZnO revealed an improvement in the injectability of the mixtures, coupled with a significant limitation in the fouling of the tools.

The formulation was carried out using natural rubber and EPDM (proportions of 70/30) for a water cistern valve part. The mould cavities are observed (24 E here with 24 cavities) and the dulled (soiled) surface on each of the cavities. The percentage expressed on the graph in FIG. 7 corresponds to the percentage of the total surface area of 24 parts contaminated as a function of time: 100% indicates that 100% of the surface of the parts is dull, and the mould totally fouled.

It will be noted in FIG. 7 that the time of use of the moulds can be doubled by using the zinc oxide according to the invention, compared with the other zinc oxides tested (HR30, CR, Bayer Aktiv).

FIGS. 2a to 2c

Diameter of particles (μm)

Diameter of particles (μm)

Diameter of particles (μm)

FIG. 3

Frequency

FIG. 4

Initial

After 7 days at 100° C. in air

Yield Strength (%)

Atomised HR30

Atomised HR30

Type and Amount of ZnO

FIG. 5

Initial

After 7 days at 100° C. in air

Initial

After 7 days at 100° C. in air

Breaking Strength (MPa)

Atomised HR30

Atomised HR30

Type and Amount of ZnO

FIG. 6

Degree of cross-linking (Points)

Reconstitution slope (%)

Atomised HR30

Atomised HR30

Type and Amount of ZnO

FIG. 7

Degree of Fouling (%)

Time (Hours)

1. Oxidised zinc compound in the form of micro-spherules consisting of smaller unitary particles having a Flodex index of less than 15.

2. Oxidised zinc compound according to claim 1, having a Flodex index of less than 10.

3. Oxidised zinc compound according to claim 1, having a compressibility index [(packed density−apparent density)×100/packed density] of less than 20%.

4. Oxidised zinc compound according to claim 3, having a compressibility index of less than 15%.

5. Oxidised zinc compound according to claim 1, having a BET specific surface of less than 100 m²/g.

6. Oxidised zinc compound according to claim 5, having a BET specific surface of less than 50 m²/g.

7. Oxidised zinc compound according to claim 1, wherein the micro-spherules have a D₅₀ (size corresponding to 50% of the total granulometric distribution curve) of between approximately 50 and approximately 200 μm.

8. Oxidised zinc compound according to claim 7, wherein the micro-spherules have a D₅₀ of between approximately 50 and approximately 100 μm.

9. Oxidised zinc compound according to claim 1, wherein the micro-spherules consist of smaller unitary particles having a D₅₀ of between approximately 1 and approximately 15 μm, without binder.

10. Oxidised zinc compound according to claim 1, selected from the group consisting of a zinc oxide, a zinc hydroxide, a zinc carbonate, a zinc hydroxycarbonate or a mixture of these.

11. Method of preparing an oxidised zinc compound in the form of micro-spherules consisting of smaller unitary particles having a Flodex index of less than 15, comprising the step of injecting, by a nozzle, an aqueous suspension of the said oxidised zinc compound, having a solid matter content of 25% to 70% by weight, at a pressure of approximately 10 to approximately 100 bar, within an atomisation chamber, in a stream of gas entering at a temperature of approximately 250° to approximately 800° C. and emerging at a temperature of approximately 50° to approximately 300° C.

12. Method according to claim 11, wherein the solid matter content is between 40% and 45% by weight in the aqueous suspension.

13. Method according to claim 11, wherein the aqueous suspension has a solid matter content of between 45% and 70% by weight, and in that a dispersing agent, such as a polyacrylate, is added to the said suspension.

14. Method according to claim 11, wherein the injection pressure is between approximately 15 and approximately 80 bar.

15. Method according to claim 11, wherein the temperature of the gas at the discharge from the atomisation chamber is between approximately 90° and approximately 220° C.

16. Method according to claim 15, wherein the temperature of the gas emerging from the atomisation chamber is between approximately 105° and approximately 170° C.

17. Method according to claim 11, wherein the gas is air.

18. Use of an oxidised zinc compound in the form of micro-spherules consisting of smaller unitary particles having a Flodex index of less than 15 in the cross-linking of polymers.

19. Use according to claim 18, in the vulcanisation of elastomer.

20. Use of an oxidised zinc compound in the form of micro-spherules consisting of smaller unitary particles having a Flodex index of less than 15 as a pigment or filler, in paints, glass or ceramics.

21. Oxidised zinc compound according to claim 7, wherein the micro-spherules have a D₅₀ of between approximately 70 and approximately 90 μm.
